(19) United States
(12) Patent Application Publication
(10) Pub. No.: US 2011/0212432 A1
(43) Pub. Date: Sep. 1, 2011

(54) SEPARATION OF BLOOD CELLS FROM A BLOOD SAMPLE

(75) Inventors: Alan Torgersen, Dundee (GB); Prabhjyot Dehal, Dundee (GB); David Pritchard, Dundee (GB); Claire Geekie, Dundee (GB)

(73) Assignee: ITI SCOTLAND LIMITED, Glasgow, GB (GB)

(21) Appl. No.: 13/060,502

(22) PCT Filed: Aug. 11, 2009
(86) PCT No.: PCT/EP2009/060356

§ 371 Date: May 4, 2011

(30) Foreign Application Priority Data
Aug. 28, 2008 (GB) 0815695.2

(51) Int. Cl. C12N 5/078; C12Q 1/02

(52) U.S. Cl. 435/2; 435/29

(57) ABSTRACT

Provided is a method for the separation of blood cells from a blood sample, which method comprises:
(a) contacting the blood sample with an agent having a binding component that binds blood cells to form bound blood cells; and
(b) separating the bound blood cells from the blood,
   wherein the agent is capable of binding a plurality of different blood cell types, and wherein the binding component is capable of binding to a protein expressed on a surface of the blood cells.

Also provided is a method for the separation of red blood cells from a blood sample, which method comprises:
(a) providing a sample collection vessel comprising an agent having a binding component that binds red blood cells;
(b) collecting a blood sample in the sample collection vessel such that red blood cells bind to the agent;
(c) removing the blood sample from the collection vessel such that red blood cells remain in the vessel;
   wherein the vessel or the agent comprises a means for retaining the red blood cells in the vessel on removal of the blood sample.

This invention relates to a method for separating blood constituents from whole blood. Specifically this invention concerns a method for removing the majority of cells from a blood sample to provide a plasma sample, using a single agent for binding all of the removed cells. The invention further provides a method for removing red blood cells by capturing the red blood cells in a sample collection vessel in advance of delivering the red cell depleted blood sample to an assay device.

A number of methods for separating red and white blood cells from blood plasma are known in the field of blood sample preparation and analysis. Traditionally, centrifugation of the blood sample is used in laboratories to separate blood cells from plasma. In the past few years improved methods for separating blood cells have been established which allow continuous separation without any use of additives.

Magnetophoretic separators target red blood cells on the basis of the paramagnetic property of deoxyhaemoglobin red blood cells and the lower magnetic susceptibility of white blood cells compared to red blood cells. “Han and Frazier (2006) Paramagnetic Capture Mode Magnetophoretic Microseparator for High Efficiency Blood Cell separations Lab on a Chip 6 265-273” describes the design, fabrication and characterization of continuous single-stage and three-stage paramagnetic capture mode magnetophoretic microseparators for high efficiency separation of blood cells. A uniform magnetic field is applied to a ferromagnetic wire, which generates a high gradient magnetic field. In a paramagnetic capture mode separator, when an external magnetic field is applied normal to a microchannel, the red blood cells, which are paramagnetic, are drawn closer to the ferromagnetic wire and white blood cells, which are diamagnetic, are forced away. This allows separation of the red blood cells from white blood cells by drawing them into separate outlet channels.

“Yang et al (2006) A microfluidic device for continuous, real time blood plasma separation Lab on a chip DOI: 10.1039/b516401j, 2006 July; 6(7): 871-80” discloses a microfluidic device for continuous, real time blood plasma separation using the principle of the blood plasma separation from blood cells by the bifurcation law, also called the Zweifach-Fung effect. When red blood cells flow through a bifurcating region of a capillary blood vessel, they travel into the daughter blood vessel which has the higher flow rate and, therefore, few cells travel into the other vessel with a lower flow rate. Blood cells are drawn into the higher flow rate vessel due to the higher pressure gradient and the torque produced on the cells, which pulls them into the faster flow rate vessel. “Yang et al (2006) A microfluidic device for continuous, real time blood plasma separation Lab on a chip DOI: 10.1039/b516401j, 2006 July; 6(7): 871-80” discloses the demonstration of a simple device containing a single bifurcation and also a device with five parallel bifurcating plasma channels. The devices were shown to successfully separate blood plasma from blood cells.

Despite the advantages discovered by the paramagnetic capture mode magnetophoretic microseparator and the microfluidic device for separating blood constituents, there is still a need to improve the speed and efficiency of blood separation methods. Whilst in “Han and Frazier (2006) Paramagnetic Capture Mode Magnetophoretic Microseparator for High Efficiency Blood Cell separations Lab on a Chip 6 265-273” and “Yang et al (2006) A microfluidic device for continuous, real time blood plasma separation Lab on a chip DOI: 10.1039/b516401j, 2006 July; 6(7): 871-80” the magnetic separator and microfluidic separator have been designed to be accurate, the flow rate of the devices was very slow and only a small volume of blood was separated.

It is an object of the present invention to overcome the problems associated with the above prior art. In particular, it is an aim of this invention to provide a method and apparatus which can separate a volume of blood at a high speed and efficiency.

Accordingly, the present invention provides a method for the separation of blood cells from a blood sample, which method comprises:
(a) contacting the blood sample with an agent having a binding component that binds blood cells to form bound blood cells; and
(b) separating the bound blood cells from the blood,
   wherein the agent is capable of binding a plurality of different blood cell types.

It is particularly preferred that the binding component is capable of binding to a protein expressed on a surface of the blood cells.

It is also particularly preferred that it is the binding component itself that is capable of binding to a plurality of different blood cell types. In this latter instance, typically an antibody is employed which is capable of binding to a protein that is present on the surface of a plurality of different cell types (preferably the majority of blood cells in a sample, as discussed below), such as an anti CD 147 antibody, or an anti CD 235 antibody.

The present invention further provides a method for the separation of red blood cells from a blood sample, which method comprises:
(a) providing a sample collection vessel comprising an agent having a binding component that binds red blood cells;
(b) collecting a blood sample in the sample collection vessel such that red blood cells bind to the agent;
(c) removing the blood sample from the collection vessel such that red blood cells remain in the vessel;
   wherein the vessel or the agent comprises a means for retaining the red blood cells in the vessel on removal of the blood sample.

Thus, typically the agent employed in the present invention is capable of binding the majority of blood cells, in order that a plasma sample can be produced, or to red blood cells so that a red blood cell depleted blood sample can be produced. It is an advantage of the present invention that the separation rate when used in devices (such as microfluidic and nanofluidic devices) is faster than in known methods, and separation is more effective and efficient. In addition, when working with small samples in such devices only a small volume of blood is available, but the efficient and speedy separation provides a greater amount of separated sample for subsequent analysis, than was previously possible.

When a plasma sample is required, it is preferred that the agent is capable of binding two, three or more of the following types of cells: red blood cells, leukocytes, platelets and endothelial cells. More preferably the agent is capable of binding all of red blood cells, leukocytes, platelets and endothelial cells. Typically, however, the agent is capable of binding the majority of the cells in the blood sample, preferably the majority of the following types of cells: erythrocytes, megakaryocytes, monocytes, macrophages, neutrophil granulocytes, eosinophil granulocytes, basophil granulocytes, mast cells, helper T cells, suppressor T cells, cytotoxic T cells, natural killer T cells, B cells, natural killer cells, dendritic cells, reticulocytes, and their stem cell progenitors. By majority, it is meant that 50% or more of the cells either by type or by total cell number are bound in a manner to allow their removal from the sample. Typically, however, cell removal is much greater than this, and 60% or more, 70% or more, 80% or more, 90% or more, 95% or more, 99% or more, 99.9% or more and even substantially 100% (substantially all) or 100% (all) of cells may be removed. Typically, even with removal of only red blood cells, 99% of cellular material has been removed. However, if it is decided only to remove other cell types, this value may be lower, depending on the cell type involved. As specific cell types can be selectively removed from retained cell fraction, the method also provides a means of rapidly sub-fractionating cell populations.

In another embodiment of the invention, when a red cell depleted blood sample is required, the agent is typically capable of binding to red blood cells, but not to other blood components (or alternatively is capable of selectively binding to red blood cells over other blood components) so that the red blood cells can be removed from the blood, thus preparing the blood for further assay. In this embodiment, the red blood cells are retained in the sample collection vessel. This vessel is not especially limited and may be any type of vessel suitable for collecting a blood sample. Preferably it is a vacutainer, or similar sample tube.

In the present method, the step of separating the bound blood cells from the blood typically comprises applying a magnetic field or an electromagnetic field, to manipulate the bound blood cells. The manipulation is not especially limited and may involve capturing and/or holding the bound blood cells (for example trapping them in a particular zone, such as in a sample collection vessel (particularly preferred) or a reaction zone, or a separation zone of a separation apparatus). When only red blood cells have been removed, remaining blood components, such as white blood cells plasma and any other blood components, may be removed in subsequent steps if desired. In an alternative embodiment, the manipulation may comprise removing the bound blood cells and leaving the remaining blood components in the sample collection vessel, separation device, reaction zone, or separation zone.

The magnetic or magnetisable substance preferably used in the present method is not especially limited. Typically it may comprise a solid surface, or most preferably a substance free to flow in a liquid, such as magnetic beads and/or magnetic proteins. When a magnetic protein is employed it may comprise a moiety for binding or encapsulating a magnetic or magnetisable substance, which moiety comprises a metal-binding protein, polypeptide, or peptide. In the present invention, typically the binding component and the magnetic protein together are comprised of a fusion protein. Preferably, the moiety for binding or encapsulating the magnetic or magnetisable substance comprises a protein, or a metal-binding domain of a protein, selected from lactoferrin, transferrin, ferritin, a ferric binding protein, frataxin, a siderophore and a metallothionein. Preferably, the moiety for binding or encapsulating the magnetic or magnetisable substance is capable of binding transition and/or lanthanide metal atoms and/or ions and/or a compound comprising such ions.

In the present invention, it is preferable that the binding component is capable of binding to a protein expressed on a surface of blood cells. However, in some instances the substance on the surface of the red blood cells may be something other than a protein. Any such substance may be employed, provided that it is capable of binding the majority of cells in blood, as described above, or is alternatively capable of distinguishing red blood cells from other components by selective binding to the binding component. Preferably, however, the protein is a transmembrane protein, such as neurothelin (CD147) (preferred for binding the majority of cells: preferably all leukocytes, red blood cells, platelets and endothelial cells) or glycophorin A (CD235a) or glycophorin B (CD235b) (each preferred for binding red blood cells). In this embodiment, it is preferred that the binding component is a neurothelin monoclonal antibody, a glycophorin A monoclonal antibody or a glycophorin B monoclonal antibody.

The present method may employ any means for separating the blood cells. However, it preferably employs a means for isolating the blood cells in a sample collection vessel. Typically this involves the attachment of the binding component to a magnetic or magnetisable surface (such as magnetic beads), the attachment of the binding component to a magnetic protein, or the attachment of the binding component to a surface of the sample collection vessel. When a magnetic bead or magnetic protein is employed, a magnetic field is applied at an appropriate point in time after binding has taken place, to ensure that the bound cells are captured in the sample collection vessel, whilst the treated sample is removed and further prepared or assayed.

The binding component employed in the present invention will now be discussed in more detail.

The binding component is not especially limited, provided that it is capable of binding to the blood cells or red blood cells as described above. Thus, the binding component may itself be any type of substance or molecule, provided that it is suitable for binding to blood cells. Generally, the binding component is selected from an antibody or a fragment of an antibody, a receptor or a fragment of a receptor, a protein, a polypeptide, a peptidomimetic, a nucleic acid, an oligonucleotide and an aptamer. In more preferred embodiments of the invention, the binding component is selected from a variable polypeptide chain of an antibody (Fv), a T-cell receptor or a fragment of a T-cell receptor, avidin, and streptavidin. Most preferably, the recognition moiety is selected from a single chain of a variable portion of an antibody (scFv).

Antibodies are immunoglobulin molecules involved in the recognition of foreign antigens and expressed by vertebrates. Antibodies are produced by a specialised cell type known as a B-lymphocyte or a B-cell. An individual B-cell produces only one kind of antibody, which targets a single epitope. When a B-cell encounters an antigen it recognises, it divides and differentiates into an antibody producing cell (or plasma cell).

The basic structure of most antibodies is composed of four polypeptide chains of two distinct types. The smaller (light) chain being of molecular mass 25 kilo-Daltons (kDa) and a larger (heavy) chain of molecular mass 50-70 kDa. The light chains have one variable (V_{L}) and one constant (C_{L}) region. The heavy chains have one variable (V_{H}) and between 3-4 constant (C_{H}) regions depending on the class of antibody. The first and second constant regions on the heavy chain are separated by a hinge region of variable length. Two heavy chains are linked together at the hinge region via disulfide bridges. The heavy chain regions after the hinge are also known as the Fc region (crystallisable fragment). The light chain and heavy chain complex before the hinge is known as the Fab (antibody fragment) region, with the two antibody binding sites together known as the F(ab)₂ region. The constant regions of the heavy chain are able to bind other components of the immune system including molecules of the complement cascade and antibody receptors on cell surfaces. The heavy and light chains of antibodies form a complex often linked by a disulfide bridge, which at the variable end is able to bind a given epitope.

The variable genes of antibodies are formed by mutation, somatic recombination (also known as gene shuffling), gene conversion and nucleotide addition events.

ScFv antibodies may be generated against a vast number of targets including:
1. Viruses: Torrance et al. 2006. Oriented immobilisation of engineered single-chain antibodies to develop biosensors for virus detection. J Virol Methods. 134 (1-2) 164-70.
2. Hepatitis C virus: Gal-Tanamy et al. 2005. HCV NS3 serine protease-neutralizing single-chain antibodies isolated by a novel genetic screen. J Mol Biol. 347 (5):991-1003), and Li and Allain. 2005 Chimeric monoclonal antibodies to hypervariable region 1 of hepatitis C virus. J Gen Virol. 86 (6) 1709-16.
3. Cancers: Holliger and Hudson. Engineered antibody fragments and the rise of single domains. Nat Biotechnol. 23 (9) 1126-36.
   and may be used in various applications including proteomics (Visintin et al. 2004. Intracellular antibodies for proteomics. J Immunol Methods. 290 (1-2):135-53).

Thus, in its most preferred embodiments, the present invention makes use of an agent, typically formed from one or more antigen binding arms of one or more antibodies, for recognising blood cells, and a solid substance such as a magnetic bead, or a magnetic protein. When the solid substance is a magnetic protein, the agent is a multi-protein agent having one or more copies of a metal-binding protein attached to the antigen binding arm of the antibody.

Typically the antibody fragment used comprises the variable regions of the heavy and light chains, V_{H} and V_{L} joined by a flexible linker to create a single chained variable fragment polypeptide, usually termed scFv. When both moieties in the agent are formed from protein and/or polypeptides (i.e. the label comprises a chimaeric protein) the label may be formed using recombinant techniques that are well known in the art. However, should any of the moieties be formed from other species, the labels may be made by simple attachment of one species to another.

Thus, the present invention also provides a method for forming a label for an analyte as defined above, which method comprises joining together a binding component for attaching the agent to red blood cells and a solid substance, such as a moiety for binding a magnetic or magnetisable substance.

The embodiment of the invention employing magnetic proteins as the magnetic or magnetisable substance will now be explained in more detail.

The moiety for binding the magnetic or magnetisable substance is not especially limited, provided that it is capable of binding the substance and does not interfere with the binding of the binding component to the blood cells. The moiety for binding the magnetic or magnetisable substance comprises a metal-binding protein, polypeptide or peptide (or the metal-binding domain of such a protein polypeptide or peptide). Typically this moiety is capable of binding to, or is bound to, one or more transition and/or lanthanide metal atoms and/or ions, or any compound comprising such ions. Such ions include, but are not limited to, any one or more ions of Fe, Co, Ni, Mn, Cr, Cu, Zn, Cd, Y, Gd, Dy, or Eu.

In the more preferred embodiments of the invention, the one or more metal ions comprise any one or more of Fe²⁺, Fe³⁺, Co²⁺, Co³⁺, Mn²⁺, Mn³⁺, Mn⁴⁺, Cd²⁺ and Ni²⁺. The most preferred ions for use in the present invention are Fe²⁺ and Fe³⁺ and Cd²⁺ and Mn²⁺ ions. Typically these ions are bound by lactoferrin, transferrin and ferritin in the case of iron, and metallothionein-2in the case of cadmium and manganese. The binding of Fe²⁺ is preferably promoted by employing acidic conditions, whilst the binding of Fe³⁺ is preferably promoted by employing neutral or alkaline conditions.

In preferred embodiments of the invention, the metal-binding moiety comprises a protein, or a metal-binding domain of a protein, selected from lactoferrin, transferrin, ferritin (apoferritin), a metallothionein (MT1 or MT2), a ferric ion binding protein (FBP e.g. from Haemophilus influenzae), frataxin and siderophores (very small peptides which function to transport iron across bacterial membranes).

In some embodiments, the labels of the invention may comprise a plurality of moieties for binding the magnetic or magnetisable substance. The number of such moieties may be controlled so as to control the magnetic properties of the label. Typically in such embodiments, the labels may comprise from 2-100 such moieties, preferably from 2-50 such moieties and most preferably from 2-20 such moieties for binding the magnetic or magnetisable substance. In the final chimaeric protein, each copy of the metal-binding protein may be attached to the next by non-charged amino acid linker sequences for flexibility.

The present invention also provides a kit for separating blood cells from blood, which kit comprises:
(a) a sample collection vessel comprising an agent having a binding component that binds blood cells; and
(b) a means for retaining the blood cells in the vessel on removal of the blood sample.

In preferred embodiments of the kits the means for retaining the blood cells in the vessel comprises attachment of the agent to a surface of the vessel, typically the majority of the inner surface of the vessel.

Alternatively, the means for retaining the blood cells in the vessel may comprise a means for magnetically influencing blood cells bound by the agent. In this embodiment, the agent comprises a binding component capable of binding blood cells, the binding component being attached to a magnetic or magnetisable substance.

In the present kits, the binding component is typically a component as described above in respect of the methods of the present invention. The magnetic or magnetisable substance is also typically one as described above in the methods of the invention.

1. A method for the separation of blood cells from a blood sample, which method comprises:
(a) contacting the blood sample with an agent having a binding component that binds blood cells to form bound blood cells; and
(b) separating the bound blood cells from the blood,
wherein the agent is capable of binding the majority of blood cell, in order that a plasma sample can be produced, and wherein the binding component is capable of binding to a protein expressed on a surface of the blood cells.

2. A method according to claim 1, wherein the agent is capable of binding two, three or more of the following types of cells: red blood cells, leukocytes, platelets and endothelial cells.

3. A method according to claim 2, wherein the agent is capable of binding red blood cells, leukocytes, platelets and endothelial cells.

4. A method according to claim 1, wherein the agent is capable of binding the majority of the following types of cells: erythrocytes, megakaryocytes, monocytes, macrophages, neutrophil granulocytes, eosinophil granulocytes, basophil granulocytes, mast cells, helper T cells, suppressor T cells, cytotoxic T cells, natural killer T cells, B cells, natural killer cells, dendritic cells, reticulocytes, and stem cells of the above.

5. A method according to claim 1, wherein the protein is a transmembrane protein.

6. A method according to claim 5, wherein the protein is an extracellular matrix metalloproteinase inducer.

7. A method according to claim 6, wherein the protein is neurothelin (CD 147).

8. A method for the separation of red blood cells from a blood sample, which method comprises:
(a) providing a sample collection vessel comprising an agent having a binding component that binds red blood cells;
(b) collecting a blood sample in the sample collection vessel such that red blood cells bind to the agent;
(c) removing the blood sample from the collection vessel such that red blood cells remain in the vessel;
wherein the vessel or the agent comprises a means for retaining the red blood cells in the vessel on removal of the blood sample.

9. A method according to claim 8, wherein the binding component is capable of binding to a protein expressed on a surface of red blood cells.

10. A method according to claim 9, wherein the protein is a transmembrane protein.

11. A method according to claim 10, wherein the transmembrane protein is glycophorin A (CD235a) or glycophorin B (CD235b).

12. The method according to any of claim 8, wherein the agent is bound to a surface of the vessel, such that red blood cells are retained in the vessel on removal of the blood sample.

13. A method according to claim 1, wherein the binding component of the agent is attached to a magnetic or magnetisable substance.

14. A method according to claim 13, wherein the step of removing the blood sample from the collection vessel comprises applying a magnetic field or an electromagnetic field to the vessel, to retain red blood cells.

15. A method according to claim 13, wherein the magnetic or magnetisable substance comprises a solid surface, magnetic beads and/or magnetic proteins.

16. A method according to claim 15, wherein the magnetic proteins comprise a moiety for binding or encapsulating a magnetic or magnetisable substance, which moiety comprises a metal-binding protein, polypeptide, or peptide.

17. A method according to claim 16, wherein the binding component and the magnetic protein together are comprised of a fusion protein.

18. A method according to claim 16, wherein the moiety for binding or encapsulating the magnetic or magnetisable substance comprises a protein, or a metal-binding domain of a protein, selected from lactoferrin, transferrin, ferritin, a ferric binding protein, frataxin, a siderophore and a metallothionein.

19. A method according to claim 16, wherein the moiety for binding or encapsulating the magnetic or magnetisable substance is capable of binding transition and/or lanthanide metal atoms and/or ions and/or a compound comprising such ions.

20. A method according to claim 19, wherein the transition metal and/or lanthanide ions comprise any one or more ions of Fe, Co, Ni, Mn, Cr, Cu, Zn, Cd, Y, Gd, Dy, or Eu.

21. A method according to claim 20, wherein the one or more metal ions comprise any one or more of Fe²⁺, Fe³⁺, Co²⁺, Co³⁺, Mn²⁺, Mn³⁺, Mn⁴⁺, Ni²⁺, Zn²⁺ and Cd²⁺.

22. A method according to claim 1, wherein the binding component is selected from an antibody or a fragment of an antibody, a receptor or a fragment of a receptor, a protein, a polypeptide, a peptidomimetic, a nucleic acid, an oligonucleotide and an aptamer.

23. A method according to claim 22, wherein the binding component is a neurothelin monoclonal antibody, a glycophorin A monoclonal antibody, or a glycophorin B monoclonal antibody.

24. A method for obtaining desired cells from a blood sample, which method comprises:
(a) a method for the separation of blood cells from a blood sample, according to a method of claim 1; and
(b) obtaining desired cells
(c) optionally isolating desired cells.

25. A kit for separating blood cells from blood, which kit comprises:
(a) a sample collection vessel comprising an agent having a binding component that binds blood cells; and
(b) a means for retaining the blood cells in the vessel on removal of the blood sample.

26. A kit according to claim 25, wherein the means for retaining the blood cells in the vessel comprises attachment of the agent to a surface of the vessel.

27. A kit according to claim 26, wherein the means for retaining the blood cells in the vessel comprises a means for magnetically influencing blood cells bound by the agent, and wherein the agent comprises a binding component capable of binding blood cells, the binding component being attached to a magnetic or magnetisable substance.

28. A kit according to any of claim 25, wherein the binding component is capable of binding to a protein expressed on a surface of blood cells.

29. A kit according to claim 28, wherein the protein is a transmembrane protein.

30. A kit according to claim 29, wherein the blood cells are red blood cells and the transmembrane protein is glycophorin A (CD235a) or glycophorin B (CD235b).

31. A kit according to claim 29, wherein the blood cells are two, three or more of red blood cells, leukocytes, platelets and endothelial cells, and the transmembrane protein is neurothelin.

32. A kit according to claim 31, wherein the blood cells are the majority of the following types of cells: erythrocytes, megakaryocytes, monocytes, macrophages, neutrophil granulocytes, eosinophil granulocytes, basophil granulocytes, mast cells, helper T cells, suppressor T cells, cytotoxic T cells, natural killer T cells, B cells, natural killer cells, reticulocytes, dendritic cells, and stem cells of the above.

33. A kit according to claim 25, wherein the binding component is selected from an antibody or a fragment of an antibody, a receptor or a fragment of a receptor, a protein, a polypeptide, a peptidomimetic, a nucleic acid, an oligonucleotide and an aptamer.

34. A kit according to claim 33, wherein the binding component is a neurothelin monoclonal antibody, a glycophorin A monoclonal antibody or a glycophorin B monoclonal antibody.

35. A method of assaying for an analyte, which method comprises:
(a) subjecting a blood sample to a method of claim 1 to form a prepared blood sample; and
(b) assaying for an analyte in the prepared blood sample.

36. A method according to claim 35, which is an in vitro diagnostic method.
